(19) United States
(12) Patent Application Publication
(10) Pub. No.: US 2008/0050755 A1
(43) Pub. Date: Feb. 28, 2008

(54) Method for screening for chemotherapeutic treatments for cancer

(76) Inventors: Nori Matsunami, Salt Lake City, UT (US); Raymond White, Salt Lake City, UT (US); Jun Liu, Salt Lake City, UT (US)

Correspondence Address: MADSON & AUSTIN, 15 WEST SOUTH TEMPLE, SUITE 900, SALT LAKE CITY, UT, 84101, US

(21) Appl. No.: 11/725,371

(22) Filed: Mar. 19, 2007

Related U.S. Application Data
(62) Division of application No. 10/312,063, filed on Dec. 20, 2002, now abandoned.
(60) Provisional application No. 60/293,357, filed on May 24, 2001.
(60) Provisional application No. 60/215,630, filed on Jun. 30, 2000.

(51) Int. Cl. G01N 33/53

(52) U.S. Cl. 435/7.21

(57) ABSTRACT

The present invention provides a method of screening for potential cancer chemotherapeutic and chemopreventive agents which act by inducing or mimicking the activity of Siah-1 in humans. This invention also provides methods of administering such agents to treat cancer by inducing or mimicking the activity of Siah-1, thus causing the degradation of .beta.-catenin.

### CROSS-REFERENCED RELATED APPLICATIONS

This application is a divisional of prior application Ser. No. 10/312,063, filed Dec. 20, 2002, which was the National Stage of International Application No. PCT/US01/20865, filed Jul. 2, 2001, which claims the benefit of U.S. Provisional Applications Nos. 60/293,357 (filed May 24, 2001) and 60/215,630 (filed Jun. 30, 2000). All of these prior applications are expressly incorporated herein by reference.

### BACKGROUND OF THE INVENTION

1. Field of the Invention

The present invention relates to chemotherapeutic treatments for cancer. More specifically, the invention relates to screening assays for cancer chemotherapeutic agents, and to novel treatments for cancer which involve the administration of agents discovered using the assays.

2. Technical Background

Cancer is a disease in which normal body cells are changed, becoming able to multiply without regard to normal cellular restraints and to invade and colonize areas of the body normally occupied by other cells. See B. Alberts et al., Molecular Biology of the Cell 1255-1294 (3d ed. 1994). According to the American Cancer Society, one-half of all American men and one-third of all American women will at some point in their lives develop cancer.

Due to the ability of cancer cells to spread and rapidly proliferate, it is difficult to treat cancer patients by attempting to selectively kill cancerous cells. Some have compared the difficulty of this task to the difficulty of completely ridding a garden of weeds. As with weeds, if only a few cancer cells are left untouched by treatment, they may again spread throughout the body, causing a recurrence of the disease. See id. at 1267. Current treatments for cancer include surgery and therapies using chemicals and radiation. The effectiveness of these treatments is often limited, however, since cancer cells that have spread from the original tumor site may be missed by surgery and radiation, and since chemical treatments which kill or disable cancer cells are often capable of causing similar damage to normal cells. See id.

Hope for better treatments for cancer focuses on obtaining a better understanding of carcinogenesis—the series of events which transforms a normal cell into a cancer cell. It is hoped that such an understanding will help researchers and physicians direct treatments solely toward cancer cells or their precursors, thus preventing or treating cancer and avoiding damage to healthy body tissues.

Inherited tumor predispositions provide an opportunity to define early, rate-limiting genetic events in the development of tumors. An inherited colon cancer predisposition, familial adenomatous polyposis, is caused by mutant alleles of the adenomatous polyposiscoli (APC) gene. White, R. L. Cell 92: 591-592 (1998). The appearance of hundreds to thousands of colon polyps among young persons affected with this disorder indicates that mutations of the APC gene can be an early and rate-limiting step in polyp development. The majority of sporadic colon polyps and carcinomas also carry mutated APC genes, establishing that somatic mutations in the APC gene are also an early event in the development of sporadic colon cancer. Powell, S. M., et al. Nature 359: 235-237 (1992).

.beta.-Catenin is a multifunctional protein that plays a role in the transduction of Wnt signals. It is also a component of the cadherin cell adhesion complex. See, Bienz, M. & Clevers, H. Cell 103: 311-320 (2000); Peifer, M., & Polalds, P. Science 287: 1606-1609 (2000). In the absence of Wnt signaling, the cytoplasmic level of .beta.-catenin is kept low through interaction with a protein complex comprised of APC, Axin, protein phosphatase 2A (PP2A), and glycogen .beta.), where .beta.-catenin is phosphorylated by GSK3.beta.. This phosphorylation of .beta.-catenin results in its ubiquitin-mediated proteasomal degradation.

Recent studies have shown that degradation of phosphorylated .beta.-catenin is triggered by specific interaction with the F-box/WD40-repeat protein, .beta.-TrCP. Winston, J. T., et al. Genes Dev. 13: 270-283 (1999); Kitagawa, M., et al. EMBO J. 18: 2401-2410 (1999); Hart, M., et al. Curr. Biol. 9: 207-210 (1999). .beta.-TrCP thus serves as an intracellular receptor for phosphorylated .beta.-catenin, forming a Skp1/Cullin/F-box protein.sup..beta.-TrCP (SCF.sup..beta.-TrCP) ubiquitin ligase complex that ubiquitinates .beta.-catenin. Activation of Wnt signaling leads to the inactivation of GSK3.beta., resulting in accumulation of cytoplasmic .beta.-catenin. At elevated cytoplasmic levels, .beta.-catenin translocates to the nucleus and cooperates with a member of the T-cell factor (Tcf)/lymphocyte enhancer-binding factor (Lef) family of high mobility group box transcription factors to activate expression of target genes.

Several lines of evidence indicate that the tumor suppressor activity of the APC protein (pAPC) relies, at least in part, on its ability to bind and subsequently down-regulate cytoplasmic .beta.-catenin. Munemitsu, S., et al. Proc. Natl Acad. Sci. USA 92: 3046-3050 (1995); Korinek, V., et al. Science 275: 1784-1787 (1997). Somatic point mutations in .beta.-catenin that alter putative GSK3.beta. phosphorylation residues have been found in several cancers. See, Bienz, M. & Clevers, H. Cell 103: 311-320 (2000), Peifer, M., & Polaks, P. Science 287: 1606-1609 (2000). These mutations weaken or abolish the association of .beta.-catenin with .beta.-TrCP and lead to an accumulation of cytoplasmic .beta.-catenin, implicating .beta.-catenin as a major etiologic target of pAPC in carcinogenesis. Mutations in the axin gene leading to the accumulation of cytoplasmic .beta.-catenin are also found in hepatocellular carcinomas. Satoh, S., et al. Nat. Genet. 24: 245-250 (2000).

An increase in cytoplasmic .beta.-catenin has been shown to promote cell proliferation and transformation, and inhibit apoptosis. Orford, K, et al. J. Cell Biol. 146: 855-868 (1999). Although both GSK3.beta. and .beta.-TrCP can participate in the pAPC-mediated down-regulation of cytoplasmic .beta.-catenin, it has been suggested that other pathways might also be important. Easwaran, V., et al J. Biol. Chem. 274: 16641-16645 (1999).

From the foregoing, it will be appreciated that it would be a significant advancement in the art to provide additional effective methods for regulating .beta.-catenin levels in cancer cells. Specifically, it would be an advancement in the art to provide methods for screening for chemotherapeutic or chemopreventive agents which act by lowering the levels of .beta.-catenin in cells, including cancer cells. It would be a further advancement in the art to provide novel treatments for cancer involving the degradation of .beta.-catenin by the administration of chemotherapeutic or chemopreventive agents discovered using such assays.

### 3. BRIEF SUMMARY OF THE INVENTION

The present invention relates to chemopreventive and chemotherapeutic treatments for cancer. More specifically, the present invention relates to methods of screening compounds for cancer chemotherapeutic activity. Said methods of screening detect agents which induce or mimic the activity of Siah-1 and thereby promote the degradation of .beta.-catenin.

In certain embodiments, the methods comprise contacting cells with a test compound, and then measuring the level of induction of Siah-1 in the cells. The induction of Siah-1 is measured in any of the numerous ways known in the art, including, but not limited to comparing the amount of Siah-1 present in a cell by western blotting with the level in untreated cells or comparing the level of .beta.-catenin in treated versus untreated cells. In certain other embodiments, said methods comprise contacting a genetically engineered cell in which p53 is suppressed with a test compound and measuring the level of Siah-1 present in the cell.

The methods of the invention can comprise the steps of contacting a cell which expresses or over-expresses .beta.-catenin with a test compound and measuring the degradation of .beta.-catenin. A compound that promotes the degradation of .beta.-catenin is a potential cancer chemotherapeutic agent. To insure that the .beta.-catenin is degraded by a compound that mimics or induces Siah-1, the activity of .beta.-TrCP and GSK3.beta., which participate in an alternative pathway for degrading .beta.-catenin, can also be suppressed in the cells used in the assays.

One may also test for Siah-1 inducing or mimicking activity of the compounds using animal models, for example in mice bearing xenografted tumors, in transgenic mice or cells derived therefrom that over-express .beta.-catenin, or in mice that are deficient in one or more of Siah-1, p53, .beta.-TrcP, and GSK3.beta.. In embodiments of the present invention, test compounds which promote the degradation of .beta.-catenin are potential cancer chemotherapeutic agents.

The invention also relates to providing novel treatments for colon cancer by administering chemotherapeutic or chemopreventive agents, discovered using the assay methods disclosed above, to a human in need of such treatment. Thus, the novel treatments for colon cancer comprise inducing Siah-1 in a human by administering a Siah-1 inducer. The treatments can also include administering a compound that mimics the activity of Siah-1 and promotes the degradation of .beta.-catenin.

As used herein, “effective amount” means an amount of a drug or pharmacologically active agent that is nontoxic but sufficient to provide the desired local or systemic effect and performance at a reasonable benefit/risk ratio attending any medical treatment. In other embodiments, the treatments comprise administering a Siah-1 inducing or mimicking compound to a human, wherein the activity of the compound does not result in significant toxic effects to the human. In yet another embodiment, the treatments comprise administering a compound which mediates the degradation of .beta.-catenin to a human in need of such treatment, wherein the ability of the compound to mediate the degradation of .beta.-catenin is determined by contacting a cell with the compound and determining whether Siah-1 is induced.

### 4. BRIEF SUMMARY OF THE DRAWINGS

FIG. 1A is a schematic representation of pAPC proteins and deletion constructs used. pAPC is a 2843-amino-acid protein that contains Armadillo repeats in the amino-terminal region, 15- and 20-aa repeats in the central region and a basic domain in the carboxy-terminal region. Near the carboxy-terminus are domains that bind to DLG, EB1 and microtubules. White, R. L. Cell 92: 591-592 (1998); Peifer, M., & Polakis, P. Science 287: 1606-1609 (2000). MCR indicates the mutation cluster region.

FIG. 1B is a schematic representation of Siah-1 proteins and deletion constructs used. The hatched box indicates the conserved RING-finger domain. .DELTA.N-Siah-1 indicates the amino-terminal deletion. .DELTA.C-Siah-1 indicates the carboxy-terminal deletion.

FIG. 1C illustrates the in vitro binding of Siah-1 to pAPC. Purified GST-pAPC fusion proteins were subjected to SDS-PAGE and detected by Commassie Brilliant Blue G staining.

FIG. 1D illustrates the in vivo binding of Siah-1 to pAPC. The Siah-1 constructs were expressed as .sup.35S-labeled proteins by in vitro transcription and translation and then incubated with the GST-pAPC fusion proteins as indicated. GST-fusion proteins were recovered on glutathione-agarose beads and subjected to SDS-PAGE. The dried gel was analyzed with a PhosphorImager. The in vitro translated (IVT) samples represent 40% of that used in the binding analysis.

FIG. 1E illustrates the in vivo binding of Siah-1 to pAPC. 293T cells were transfected with a plasmid expressing Myc epitope-tagged Siah-1 or an empty expression vector. The whole cell extract (WCE) was immunoprecipitated using a mouse monoclonal antibody specific for the amino-terminus of pAPC or a control mouse IgG, and associated Myc-Siah-1 protein was detected by immunoblotting using an anti-Myc monoclonal antibody. The pAPC in WCE (20 .mu.g) and immunoprecipitates were detected using an anti-APC mouse monoclonal antibody.

FIGS. 2A, 2B, and 2C are bar graphs illustrating the effect of Siah-1 on Tcf/Lef reporter activity, .beta.-catenin levels and turnover. 293T cells were co-transfected with a reporter construct (pTOPFLASH or pFOPFLASH), an internal control (pCMV.beta.-gal) and indicated plasmids. Korinek, V., et al. Science 275: 1784-1787 (1997); Morin, P. J., et al. Science 275: 1787-1790 (1997). The amount of DNA in each transfection was kept constant by the addition of an appropriate amount of empty expression vector. Luciferase and .beta.-galactosidase activity was measured 24 hours after transfecton. Tcf/Lef reporter activity was determined as described. Korinek, V., et al. Science 275: 1784-1787 (1997); Morin, P. J., et al. Science 275: 1787-1790(1997). The result is shown as relative Tcf/Lef reporter activity (TOP/FOP). The histograms are presented as the average.±.SD from multiple experiments. FIG. 2A shows that Siah-1 down-regulates .beta.-catenin-induced Tcf/Lef reporter activity in a dose dependent manner. FIG. 2B shows that DN-Siah-1 up-regulates Tcf/Lef reporter activity acting as a dominant-negative form. FIG. 2C shows that Siah-1 down-regulates Tcf/Lef reporter activity induced by soluble Wnt-3a conditioned medium.

FIG. 2D illustrates the transient co-transfection of 293T cells were with an internal control (pEGFP) and the indicated plasmids. The amount of DNA in each transfection was kept constant by the addition of an appropriate amount of empty expression vector. Whole cell lysates were subjected to Western analysis. Blots were probed with mouse monoclonal antibodies to Myc epitope tag (upper panel) and GFP (lower panel).

FIG. 2E illustrates the pulse-chase analysis of ectopically expressed Myc-tagged .beta.-catenin. 293T cells were transiently co-transfected with the indicated plasmids, pulse-labeled with .sup.35S-methionine and cysteine, and then chased with media lacking the labeled amino acids. Cells were lysed at the indicated times and the expressed Myc-.beta.-catenin was recovered by immunoprecipitation via a Myc epitope tag. Immunoprecipitated Myc-.beta.-catenin was subjected to SDS-PAGE and dried gels were analyzed with a PhosphorImager. The blots shown are representative of multiple experiments.

FIGS. 3A, 3B, and 3C show that Siah-1 down-regulates .beta.-catenin through a mechanism independent of GSK3.beta.-mediated phosphorylation and the .beta.-TrCP-mediated proteasome pathway. 293T cells were co-transfected with an internal transfection efficiency control (pEGFP) and the indicated plasmids. The amount of DNA in each transfection was kept constant by the addition of an appropriate amount of empty expression vector. Whole cell lysates were subjected to Western analysis. Blots were probed with mouse monoclonal antibody recognizing the FLAG-tag, HA-tag, Myc-tag or GFP. The blots shown are representative of multiple experiments. FIG. 3A show that the over-expression of GSK3.beta. down-regulates wild-type .beta.-catenin but not mutant .beta.-catenin which has substitutions of GSK3.beta. phosphorylation sites. FIG. 3B shows that Siah-1 can down-regulate both wild-type and mutant .beta.-catenin. WT indicates wild-type and Mut indicates mutant. FIG. 3C show that dominant-negative (DN) .beta.-TrCP does not block Siah-1-mediated down-regulation of wild-type .beta.-catenin.

FIGS. 4A and 4B are bar graphs showing that pAPC interacts with Siah for down-regulation of Tcf/Lef reporter activity. Cells were co-transfected with a reporter construct (pTOPFLASH or pFOPFLASH), an internal control (pCMV.beta.-gal) and indicated plasmids. Korinek, V., et al. Science 275: 1784-1787 (1997); Morin, P. J., et al. Science 275: 1787-1790 (1997). The amount of DNA in each transfection was kept constant by the addition of an appropriate amount of empty expression vector. Luciferase and .beta.-galactosidase activities were measured 24 hours after transfection. Tcf/Lef reporter activity was determined as described and the result is shown as relative Tcf/Lef reporter activity (TOP/FOP). Korinek, V., et al. Science 275: 1784-1787 (1997); Morin, P. J., et al. Science 275: 1787-1790 (1997). The histograms are presented as the average.±.SD from multiple experiments. As shown in FIG. 4A, Siah-1 downregulates Tcf/Lef reporter activity in LS174T colon cancer cells containing wild-type pAPC and mutant .beta.-catenin, but it has no effect in colon cancer cell lines DLD-1 and SW480 containing truncated mutant pAPC and wild-type .beta.-catenin. FIG. 4B shows that the carboxy terminal pAPC fragment (a.a. 2543-2843) blocks Siah-l-mediated down-regulation of Tcf/Lef reporter activity in 293T cells.

FIGS. 5A, 5B, 5C, and 5D, and 5E show that Siah-1 induces reduced dorsoanterior development in Xenopus embryos and counteracts Wnt-8 signaling. FIG. 5A shows an embryo with normal dorsoanterior development. FIG. 5B shows that an embryo injected with RNA encoding Siah-1 in the dorsal side has reduced dorsoanterior development, including loss of eyes and reduced head structures. In contrast, FIG. 5C shows that embryos injected with RNA encoding Xwnt-8 have enlarged dorsoanterior structures. FIG. 6D show that co-injection of RNAs encoding Siah-1 and Xwnt-8 results in fairly normal embryos, with eyes and well-developed head structures. In all panels, dorsal is to the top and anterior is to the left. FIG. 5E is a chart summarizing the data obtained from the injection experiments. The dorso-anterior index (DAI) was scored by the method of Kao and Elinson (1988). Kao, K. R. & Elinson, R. P. Dev. Biol. 127: 64-77 (1988).

FIGS. 6A, 6B, 6C, and 6E illustrate that Siah-1 mediates p53-induced degradation of .beta.-catenin. FIG. 6A shows that p53 induces Siah-1 transcript in 293T cells. FIG. 6B shows the effect of p53, p21 and the carboxy-terminal pAPC peptide on the amount of .beta.-catenin. 293T cells were transiently co-transfected with an internal control (pEGFP) and the indicated plasmids. The amount of DNA in each transfection was kept constant by the addition of an appropriate amount of empty expression vector. Whole cell lysates were subjected to Western analysis. Blots were probed with mouse monoclonal antibodies to the Myc epitope tag (upper panel) and GFP (lower panel). FIG. 6C illustrate the pulse-chase analysis of ectopically expressed Myc-tagged .beta.-catenin. 293T cells were transiently co-transfected with the indicated plasmids, pulse-labeled with .sup.35S-methionine and cysteine, and then chased with media lacking the labeled amino acids. Cells were lysed at the indicated times and the expressed Myc-.beta.-catenin was recovered by immunoprecipitation via a Myc epitope tag. Immunoprecipitated Myc-.beta.-catenin was subjected to SDS-PAGE and dried gels were analyzed with a PhosphorImager. FIG. 6D shows the effect of .DELTA.N-Siah-1 and the carboxy-terminal pAPC peptide on p53-mediated down-regulation of .beta.-catenin. FIG. 6E is a bar graph showing the effect of adriamycin and the carboxy-terminal pAPC peptide on Tcf/Lef reporter activity. The amount of DNA in each transfection was kept constant by the addition of an appropriate amount of empty expression vector. 293T cells were treated with 1 .mu.g/ml adriamycin 8 hours after transfection for 16 hours. The blots shown are representative of multiple experiments. The histograms are presented as the average.±.SD from multiple experiments.

These drawings only provide information concerning typical embodiments of the invention and are not therefore to be considered limiting of its scope.

### 5. DETAILED DESCRIPTION OF THE INVENTION

The present invention provides screening assays for cancer chemotherapeutic agents which induce or mimic the activity of the Siah-1 in promoting the degradation of .beta.-catenin. The invention also provides novel treatments for colon cancer which involve the degradation of .beta.-catenin by the administration of chemotherapeutic or chemopreventive agents discovered using such assays.

All publications, patents, and patent applications cited herein are hereby incorporated by reference.

Definitions

By “vector” is meant any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors.

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al., 52 Virology 456 (1973); Sambrook et al., Molecular Cloning: A Laboratory Manual, Cold Spring Harbor Laboratories, New York (1989); Davis et al., Basic Methods in Molecular Biology, Elsevier, (1986); Chu et al., 13 Gene 197 (1981); and J. D. Watson et al., Recombinant DNA, 213-234 (1996). Such techniques can be used to introduce one or more exogenous DNA moieties, such as a nucleotide integration vector and other nucleic acid molecules into suitable host cells. The term captures chemical, electrical, and viral-mediated transfection procedures, including, as examples, calcium phosphate co-precipitation (Graham et al., 52 Virology 456-467 (1973)), direct micro-injection into cultured cells (M. R. Capecci, 22 Cell 479-488 (1980)), electroporation (Shigekawa et al., 6 BioTechniques 742-751 (1988)), liposome-mediated gene transfer (Mannino et al., 6 BioTechniques 682-690 (1988)), lipid-mediated transfection (Felgner et al., 84 Proc. Natl. Acad. Sci. 7413-7417 (1987)), and nucleic acid delivery using high-velocity microprojectiles (Klein et al., 327 Nature 70-73 (1987)).

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

The term “amplicon” is used to mean a segment of genetic material which forms many linear copies upon exposure to a compound which inhibits the function of a gene found in the segment.

The term “over-expression” as used herein denotes that a given gene product is expressed in a cell or set of cells that have been engineered to express the gene product at a rate higher than in a comparable cell or set of cells that have not been so engineered. The rates of over-expression in said cells vary from the original levels by 2-fold, 5-fold, and 10-fold, with 10-fold being preferred.

The term “suppressed” as used herein denotes that a given gene product is present in a cell, a set of cells, or an animal at a rate lower than the normal wild type level. Suppression may occur from a naturally occurring or engineered mutation to the gene or by contacting the cell, set of cells, or the animal with a compound that is known to suppress the expression of the gene or inactivate the gene product. Suppression also refers to the state when none of a given gene product is present in the cell, cell line, or animal.

General Methods

The invention stems from the discovery that Siah-1, the human homologue of Drosophila seven in absentia, is a p53-inducible mediator of cell cycle arrest, tumor suppression and apoptosis which promotes degradation of .beta.-catenin. Siah-1 interacts with the carboxy-terminus of APC and promotes degradation of .beta.-catenin in mammalian cells. The ability of Siah-1 to down-regulate .beta.-catenin signaling was also demonstrated by hypodorsalization of Xenopus embryos. Unexpectedly, degradation of .beta.-catenin by Siah-1 was independent of GSK3.beta.-mediated phosphorylation and did not require the F-box protein .beta.-TrCP. These data indicate that APC and Siah-1 mediate a novel .beta.-catenin degradation pathway linking p53 activation to cell cycle control.

In order to discover compounds which induce degradation of .beta.-catenin via Siah-1, method of screening test compounds can use human epithelial cell or other cell lines. Such methods comprise contacting a test composition with a cell and measuring the induction of Siah-1 in the cell relative to that of a non-contacted cell. Many methods may be used to measure the induction of Siah-1 including tracking the degradation of .beta.-catenin in the cell compared to non-treated cells. Similarly, pulse-chase methods such as that used in Example 3 can show relative amounts of .beta.-catenin degradation in a set of cells versus that of a control. Further, the amount of Siah-1 in a test cell can be determined by western blot methods and compared to a control.

The cells used to test the compound can be from various sources such as epithelial cells, fibroblasts, epithelial tumor cells, colorectal cancer cells, and genetically engineered cells. Cells may be genetically engineered for suppression Siah-1, p53, .beta.-TrCP, and/or Gsk3.beta..

Animal models for cancer such as mice can also be used for the screening methods of the present invention. Such methods include administering a test compound to the animal and measuring the induction of Siah-1 in the animal. When a test compound induces Siah-1 the compound maybe used to treat cancer by administering a therapeutically effective amount of the compound to promote the degradation of .beta.-catenin. The test compound may also mimic the activity of Siah-1. By mimicking the activity of Siah-1, it is meant that the compound promotes the degradation of .beta.-catenin independent of .beta.-TrCP, and Gsk3.beta..

The animal model can be a mouse in which the expression of Siah-1, p53, .beta.-TrCP, and/or Gsk3 .beta. is suppressed. Moreover, to more easily determine the effect of the compound on .beta.-catenin levels, the animal or cell can be engineered to overexpress .beta.-catenin. The animal model may also be a mouse bearing a xenografted tumor.

The test compounds that are determined to induce the activity of Siah-1 in degrading .beta.-catenin can be used to treat a human suffering from cancer. Moreover, the compounds that mimic the activity of Siah-1 by promoting the degradation of .beta.-catenin independent of .beta.-TrCP and Gsk3.beta. can also be used to treat cancer in a human.

In order to better describe the details of the present invention, the following discussion is divided into three sections: (1) APC and Siah-1 present an alternative pathway for targeted destruction of .beta.-catenin; (2) role of p53 in Siah-1-mediated regulation of .beta.-catenin turnover and cell growth.; and (3) Role of Siah-1 in colon carcinogenesis.

5.1 APC and Siah-1: an Alternative Pathway for Targeted Destruction of .beta.-catenin

The invention stems from the discovery that Siah-1 binds the carboxy-terminus of pAPC both in vitro and in vivo. Expression of Siah-1 reduces the abundance of .beta.-catenin protein and inhibited both .beta.-catenin- and Wnt-3a-induced Tcf/Lef-dependent transcriptions. The ability of Siah-1 to down-regulate .beta.-catenin signaling was also shown in Xenopus embryos, inducing hypodorsalization and counteracting Xwnt-8. Pulse-chase analysis indicated that expression of Siah-1 promoted down-regulation of .beta.-catenin in a post-translational manner. Expression of the amino-terminal deletion mutant, .DELTA.N-Siah-1, known to function as a trans-dominant inhibitor of endogenous Siah-1 increased Tcf/Lef reporter activity and stabilized .beta.-catenin in the pulse-chase experiment, suggesting that endogenous Siah-1 also down-regulates .beta.-catenin. Hu, G. & Fearon, E. R. Mol. Cell. Biol. 19: 724-732 (1999).

Unexpectedly, Siah-1 induced ubiquitination and down-regulation of both wild-type and non-phosphorylatable mutant .beta.-catenin. Over-expression of a dominant-negative .beta.-TrCP failed to block Siah-1-mediated down-regulation of .beta.-catenin. A requirement for pAPC was indicated by the failure of Siah-1 to decrease Tcf/Lef reporter activity in the colon cancer cell lines SW480 and DLD1 which express only truncated pAPC, but not in LS174T colon cancer cells which express a wild-type pAPC. Smith, K. J., et al. Proc. Natl Acad. Sci. USA 90: 2846-2850 (1993).

Other evidence for a requirement for pAPC was indicated by co-expression of the carboxy-terminal peptides of pAPC which inhibited Siah-1-mediated down-regulation of Tcf/Lef reporter activity. These results indicate that Siah-1, together with pAPC, mediates degradation of .beta.-catenin through a novel mechanism, independent of both GSK3.beta.-mediated phosphorylation and the .beta.-TrCP-mediated proteasome pathway.

Although APC and Siah-1 can participate in the down-regulation of both wild-type and mutant .beta.-catenins and Siah-1 can counteract Wnt signaling in both mammalian cells and Xenopus laevis embryos, the relationship of the pAPC/Siah-1-mediated pathway for controlling .beta.-catenin levels compared to the classical Wnt pathway is unclear. A new pathway was recently mapped which links Siah-1 to a new Siah-interacting protein (SIP), another F-box protein (Ebi), Skp1 and .beta.-catenin SIP binds simultaneously to Siah-1 and to Skp1 serving as a molecular bridge to unite two proteins. SIP also associates with SCF complexes containing Skp1 and Ebi, but is not found in SCF complexes containing .beta.-TrCP. Furthermore, while Ebi and .beta.-TrCP can both bind wild-type .beta.-catenin, only Ebi is capable of binding mutant .beta.-catenin. Thus, Siah-1, APC, SIP, Skp1, and Ebi collaborate in a novel pathway for controlling turnover of both wild-type and mutant .beta.-catenins, and affect activity of .beta.-catenin-dependent transcription.

5.2 Role of p53 in Siah-1-Mediated Regulation of .beta.-catenin Turnover and Cell Growth.

In mammalian cells, Siah-family proteins are p53-inducible mediators of cell cycle arrest, tumor suppression and apoptosis. Amson, R. B. et al. Proc. Natl Acad. Sci. USA 93: 3953-3957 (1996); Nemani, M. et al. Proc. Natl Acad. Sci. USA 93: 9039-9042 (1996); Hu, G., et al. Genomics 46:103-111(1997); Matsuzawa, S., et al. EMBO J. 17: 2736-2747 (1998); Linares-Cruz, G. et al. Proc. Natl Acad. Sci. USA 95: 1131-1135 (1998); Roperch, J. P., et al. Proc. Natl Acad. Sci. USA 96: 8070-8073 (1999). Siah proteins target the ubiquitin-mediated proteolysis of the DCC (deleted in colon cancer), N-CoR (nuclear receptor co-repressor) and c-Myb proteins. Hu, G., et al. Genes Dev. 11: 2701-2714 (1997); Zhang, J., et al. Genes Dev. 12: 1775-1780 (1998); Tanikawa, J., et al. J. Biol. Chem. 275: 15578-1558 (2000). Because Siah proteins have a short half-life and are normally maintained at a relatively low level, activation of p53 by genotoxic reagents or radiation would induce Siah and trigger destruction of target proteins. Hu, G. & Fearon, E. R. Mol. Cell. Biol. 19: 724-732 (1999). Similarly, mutational inactivation of the p53 gene may prevent Siah induction, leading to a failure of Siah-mediated down-regulation of target proteins. .beta.-catenin activates expression of target genes to promote the G1 to S phase transition and inhibit apoptosis. Bienz, M. & Clevers, H. Cell 103: 311-320 (2000); Peifer, M., & Polakis, P. Science 287: 1606-1609 (2000). .beta.-catenin also attenuates the G1 to S phase cell cycle block induced by radiation. Orford, K, et al. J. Cell Biol. 146: 855-868 (1999). Taken together, these findings suggest .beta.-catenin as aplausible target of a Siah-1-mediated protein degradation pathway linking p53-activation to cell cycle control. Interestingly, UV radiation induces ventralization in Xenopus laevis embryos, a similar phenotype observed in the Siah-1 microinjection experiments. Smith, W. C. & Harland, R. M. Cell 67: 753-765 (1991).

Although little is known about the regulation of Siah-1 by p53, expression of Siah-1 was also induced following introduction of the cyclin-dependent kinase inhibitor p21, a mediator of p53-induced G1-arrest), suggesting p21 as a downstream activator of p53 for Siah-1 expression. Linares-Cruz, G. et al. Proc. Natl Acad. Sci. USA 95: 1131-1135 (1998); Roperch, J. P., et al. Proc. Natl Acad. Sci. USA 96: 8070-8073 (1999). The finding that both p53 and p21 can reduce the amount of .beta.-catenin protein supports this view.

5.3 Role of Siah-1 in Colon Carcinogenesis

The majority of APC mutations associated with intestinal polyps and carcinomas are found between codons 1250 and 1450, in the mutation cluster region (MCR). Such mutations produce truncated proteins unable to bind and down-regulate the cytoplasmic levels of .beta.-catenin through GSK3.beta.-mediated pathway. Munemitsu, S., et al. Proc. Natl Acad. Sci. USA 92: 3046-3050 (1995); Korinek, V., et al. Science 275: 1784-1787 (1997);Morin, P. J., et al. Science 275: 1787-1790 (1997). In addition, these truncated proteins lack the carboxy-terminal domain that would bind DLG, EB1, microtubules and Siah-1. Peifer, M., & Polakis, P. Science 287: 1606-1609 (2000).

Several mouse models have been reported with specific mutations in the 5′ region of the corresponding MCR of the murine Apc gene. Su, L. K., et al. Science 256: 668-670 (1992); Fodde, R., et al. Proc. Natl. Acad. Sci. USA 91: 8969-8973 (1994); Oshima, M., et al. Proc. Natl. Acad. Sci. 92: 4482-4486 (1995). Mice heterozygous for such Apc mutations develop intestinal polyps and tumors. Homozygous mice die during embryogenesis before day 8 of gestation, suggesting a role for Apc during early development. Fodde, R., et al. Proc. Natl. Acad. Sci. USA 91: 8969-8973 (1994); Oshima, M., et al. Proc. Natl. Acad. Sci. 92: 4482-4486 (1995). A mutation was introduced downstream of the murine MCR at codon 1638 (1638T). Smits, R et al. Genes Dev. 13: 1309-1321 (1999). This mutation would produce a truncated Apc protein that does not have the carboxyl-terminal domains, but is still able to bind and down-regulate cytoplasmic .beta.-catenin. Homozygous and heterozygous animals were viable and tumor free, suggesting that the deleted carboxyl-terminal domains of Apc are less essential for both embryonic development and tumor suppression. Smits, R. et al. Genes Dev. 13: 1309-1321 (1999).

However, both germline and somatic mutations have been reported beyond codon 1638 of the APC gene in polyposis patients. These mutations would produce truncated proteins that should still be able to down-regulate .beta.-catenin through GSK3.beta.-mediated pathway but should not bind Siah-1. Unlike the 1638T mice, humans carrying such 3 mutations do show a polyposis phenotype. One possible explanation is that they might not produce detectable levels of truncated proteins, possibly due to decreased stability. van der Luijt, R. B., et al. Hum. Geizet. 98: 727-734 (1996). Another possible explanation is that, at least in human, the carboxy-terminus of pAPC may carry additional tumor suppressing activities, and its loss increases the risk of cancer.

Interestingly, .beta.-catenin mutations are only rarely seen in invasive colon carcinomas, and small adenomas with .beta.-catenin mutations do not appear to be as likely to progress to larger adenomas and invasive carcinomas as other adenomas. Samowitz, W. S., et al. Cancer Res. 59: 1442-1444 (1999). This suggests that APC and .beta.-catenin mutations are not functionally equivalent and that pAPC may have other tumor suppressor functions. Samowitz, W. S., et al. Cancer Res. 59: 1442-1444 (1999).

The finding that Siah-1 interacts with the carboxy-terminus of pAPC and down-regulates both wild-type and mutant .beta.-catenins supports this view since mutations in the MCR of the APC gene would prevent .beta.-catenin degradation mediated by both Wnt pathway and Siah-1. Mutant .beta.-catenins, however, would still be prone to Siah-1-mediated degradation in the presence of wild-type pAPC. Furthermore, mutational inactivation of the p53 gene, commonly seen in various types of cancers, might prevent Siah induction, leading to a failure of Siah-mediated down-regulation of .beta.-catenin, and cooperate with mutations in Wnt-pathway components such as axin and .beta.-catenin.

### 6. EXAMPLES

The following examples are given to illustrate various embodiments which have been made with the present invention. It is to be understood that the following examples are not comprehensive or exhaustive of the many types of embodiments which can be prepared in accordance with the present invention.

Materials and Methods

Plasmids. Plasmids expressing Siah-1, .DELTA.N-Siah-1 and .DELTA.C-Siah-1 were described previously. Matsuzawa, S., et al. EMBO J. 17: 2736-2747 (1998). For production of GST-pAPC fusion constructs, DNA cassettes encoding the carboxy-terminal pAPC were PCR amplified and inserted in frame into downstream of GST gene in pGEX-2TK (Pharmacia) using standard PCR cloning techniques. The primers used to amplify pAPC (a.a. 2688-2843) were APC2688 (5′-GCGCGC GGATCC ATGGAA AAGGCA AATCCA AACATT-3′) (SEQ ID NO 1) and RAPC (5′-GGCCGG GGATCC TTAAAC AGATGT CACAAG GTAAGA-3) (SEQ ID NO 2). The primers for pAPC (a.a. 2543-2843) were APC2543 (5′-GCGCGC GGATCC ATGTCA GGAACC TGGAAA CGTGAG-3) (SEQ ID NO 3) and RAPC. To express the carboxy-terminal pAPC peptides in mammalian cells, DNA cassette encoding the carboxy-terminal pAPC (aa. 2543-2843) was amplified and inserted into pcDNA3 using standard PCR cloning techniques with primers E-APC2543 (5′-GCGCGC GGATCC GCCGCC ACCATG GACTAC AAGGAC GACGAT GACAAG TCAGGA ACCTGG AAACGT GAG-3′) (SEQ ID NO 4) and RAPC. A plasmid expressing Myc-tagged .beta.-catenin was kindly provided by Dr. P. Polakis. For generating plasmids encoding Flag-tagged .beta.-catenins, primers KPNFBCAT (5′-GCCAGT GGTACC GCCGCC ACCATG GATTAC AAGGAT GACGAC GATAAG GCTACT CAAGCT GATTTG-3′) (SEQ ID NO 5), containing the Kozak sequence and FLAG-tag, and XBABCAT (5′-ACAGCT ATGACC TCTAGA TTACAG GTCAGT ATCAAA CCAGG-3′) (SEQ ID NO 6) were first used to PCR amplify the wild-type .beta.-catenin. To construct Flag-tagged mutant .beta.-catenin, which has phenylalanine and alanine substitutions in putative GSK3.beta. phosphorylation sites (serine and threonine amino acid residues between 33 and 45), DNA fragments encoding the amino- and the carboxy-terminal regions were separately PCR amplified with primer sets, KPNFBCAT and MutTACB (5′-AAAAGG AGCTGT GGCAGT GGCACC AAAATG GATTCC AAAGTC CAGGTA AGACTG TTG-3) (SEQ ID NO 7), and XBABCAT and MutBCAT (5′-TTTGGA ATCCAT TTTGGT GCCACT GCCACA GCTCCT TTTCTG AGTGGT AAAGGC AAT-3′) (SEQ ID NO 8), respectively. The two PCR products were mixed and subjected to the second PCR reaction to obtain the full-length mutant .beta.-catenin. Both wild-type and mutant .beta.-catenin DNA fragments were digested with KpnI and BamHI, and cloned into expression vector pcDNA. For dominant-negative .beta.-TrcP, wild-type .beta.-TrCP cDNA was first amplified by RT-PCR from mRNA of colon cancer cell line SW480 using primers F1 (5′-GCGCGC GGATCC GCCGCC ACCATG GACTAC AAGGAC GACGAT GACAAG GACCCG GCCGAG GCGGTG CTG-3′) (SEQ ID NO 9), containing the Kozak sequence and FLAG-tag, and R1 (5′-GGCCGG TCTAGA TTATCT GGAGAT GTAGGT GTATGT-3′) (SEQ ID NO 10). The dominant-negative .beta.-TrCP, which does not have F-box, was constructed as follows. The amino-terminal region of .beta.-TrCP (a.a. 1-147) was amplified with primers F1 and R2 (5′-GGCCGG CTCGAG AGCAGT TATGAA ATCTCT CTG-3′) (SEQ ID NO 11), and was digested with BamHI and XhoI. The carboxy-terminal region of .beta.-TrCP (a.a. 193-569) was amplified with primers F2 (5′-GCGCGC CTCGAG AGAATG GTCAGG ACAGAT-3′) (SEQ ID NO 12) and R1, and was digested with XhoI and XbaI. The two fragments were ligated into BamHI/Xbal sites of pcDNA. All constructs were confirmed by DNA sequencing.

In vitro protein binding assay. APC cDNAs encoding the carboxy terminus (a.a. 2688-2843 and a.a. 2543-2843) were expressed in DH5.alpha. cells (Life Technologies), and affinity-purified using glutathione-Sepharose. Purified GST fusion proteins (10 .mu.g) and 5 .mu.l of rabbit reticulocyte lysates (TNT coupled reticulocyte lysate system; Promega) containing .sup.35S-labeled, in vitro translated (IVT) Siah-1 proteins were incubated in 100 .mu.l of buffer containing 25 mM Tris (pH 7.5), 50 mM NaCl, 1 mM dithiothreitol, 0.1% NP-40 and 0.1 mg/ml bovine serum albumin at 4.degree. C. for 2 hours. GST-fusion proteins were recovered on glutathione-Sepharose beads. The beads were washed four times with L-buffer (PBS, 0.1% NP-40 and 0.1% Triton X-100) and boiled in Laemmli-SDS sample buffer. The eluted proteins were subjected to SDS-PAGE and the dried gel was analyzed with a PhosphorImager (Molecular Dynamics).

Cell culture, transfection, reporter assay, co-immunoprecipitation and immmunoblotting. 293T cells were maintained in DMEM supplemented with 10% fetal bovine serum (FBS). Colon cancer cell lines DLD-1, SW480, and LS174T were cultured in RPMI1640-10% FBS, Leibovitz's L-15-10% FBS and MEM-10% FBS, respectively. DNA transfection experiments were carried out by calcium phosphate method. The amount of DNA in each transfection was kept constant by the addition of an appropriate amount of empty expression vector. To determine Tcf/Lef reporter activity, subconfluent cells were co-transfected with a reporter construct (0.5 .mu.g of pTOPFLASH or pFOPFLASH), an internal control (0.05 .mu.g of pCMV.beta.-gal) and indicated plasmids in six-well plates. Korinek, V., et al. Science 275: 1784-1787 (1997); Morin, P. J., et al. Science 275: 1787-1790 (1997). Luciferase and .beta.-galactosidase activity was measured 24 hours after transfecton. Tcf/Lef reporter activity was determined as described. Korinek, V., et al. Science 275: 1784-1787 (1997); Morin, P. J., et al. Science 275: 1787-1790 (1997). Co-immunoprecipitation was performed as previously described. Neufeld, K. L., et al. EMBO Reports 1: 519-523 (2000). The whole cell extract (WCE) was immunoprecipitated using a mouse monoclonal antibody specific for the amino-terminus of pAPC (Ab-5, Oncogene Research) or a control mouse IgG, and associated Myc-Siah-1 protein was detected by immunoblotting using an anti-Myc monoclonal antibody (9E10). The pAPC in WCE (20 .mu.g) and immunoprecipitates was detected using an anti-APC mouse monoclonal antibody (Ab-1, Oncogene Research). To detect Myc-.beta.-catenin, FLAG-.beta.-catenin, HA-GSK3 .beta. and GFP proteins, transfected cells were lysed directly in Laemmli-SDS sample buffer 48 hours after transfection. Total cellular proteins (40 .mu.g/lane) were separated by 4-20% gradient Tris-glycine SDS-PAGE and were transferred to nitrocellulose membrane. Proteins were detected with primary antibodies and horseradish peroxidase-conjugated secondary antibodies using an ECL system (Amersham) and PhosphorImager (Molecular Dynamics). Primary antibodies used were mouse anti-c-Myc monoclonal antibody (9E10, Santa Cruz Biotechnology), mouse anti-FLAG monoclonal antibody (M2, Sigma), mouse anti-HA monoclonal antibody (HA.11, Babco), mouse anti-GFP monoclonal antibody (Clontech) and mouse anti-ubiquitin monoclonal antibody (P1A6, Santa Cruz Biotechnology). Blots were analyzed with a Lumi-Imager (Boehringer Mannheim).

Pulse-chase analysis. To perform pulse-chase analysis of ectopically expressed Myc-tagged .beta.-catenin, 293T cells were transiently transfected in six-well plates. 36 hours after transfection, cells were pulse-labeled for one hour with 0.1 mCi of .sup.35S-methionine and cysteine per each well, and then chased with cold media. Cells were lysed in RIPA buffer (0.05M Tris buffer, pH7.2, 0.15M NaCl, 1% Triton-X100, 1% deoxycholate, 0.1% SDS) supplemented with protease inhibitors at the indicated times. After centrifugation of the lysates, Myc-.beta.-catenin was immunoprecipitated from supernatants by mouse anti-c-Myc monoclonal antibody (9E10) conjugated to agarose (Santa Cruz Biotechnology). After three washes with RIPA buffer, immunoprecipitates were subjected to SDS-PAGE. Dried gels were analyzed with a PhosphorImager (Molecular Dynamics).

Microinjection of Xenopus Embryos. RNA was microinjected into the dorsal side of 4-cell-stage Xenopus embryos. The amount of RNA used for injections (in 5 nl) were 1.5 ng for Siah-1 and 12.5 pg for Xwnt-8. The average dorso-anterior index (DAI) was scored by the method of Kao and Elinson (1988). Kao, K. R. & Elinson, R. P. Dev. Biol. 127: 64-77 (1988).

### Example 1

pAPC Interacts with Siah-1

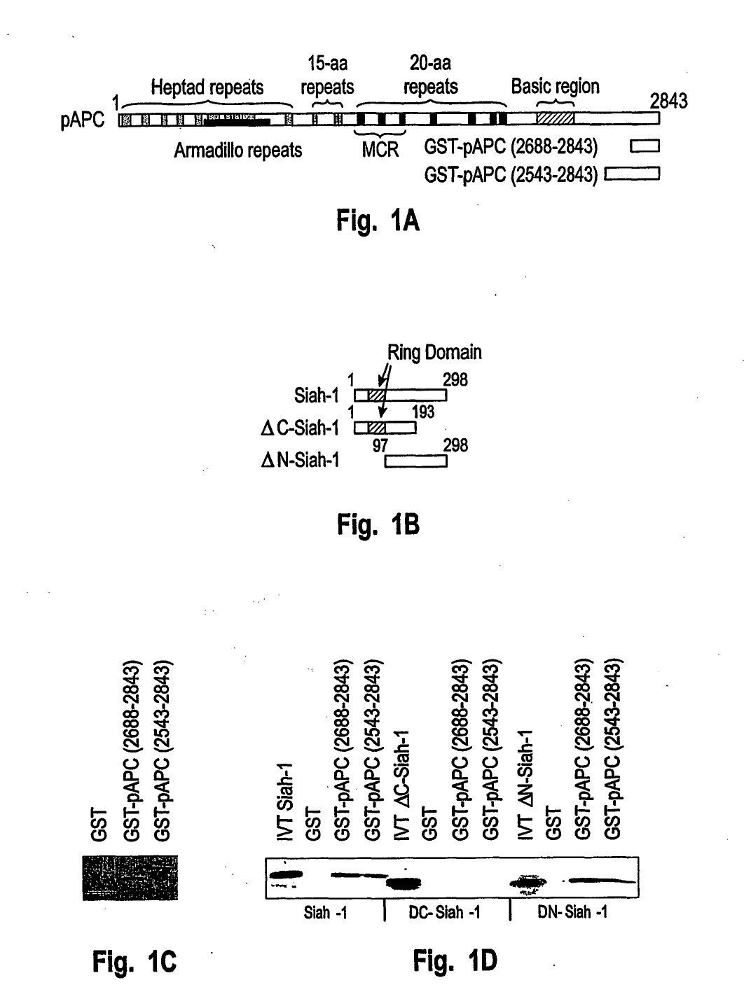

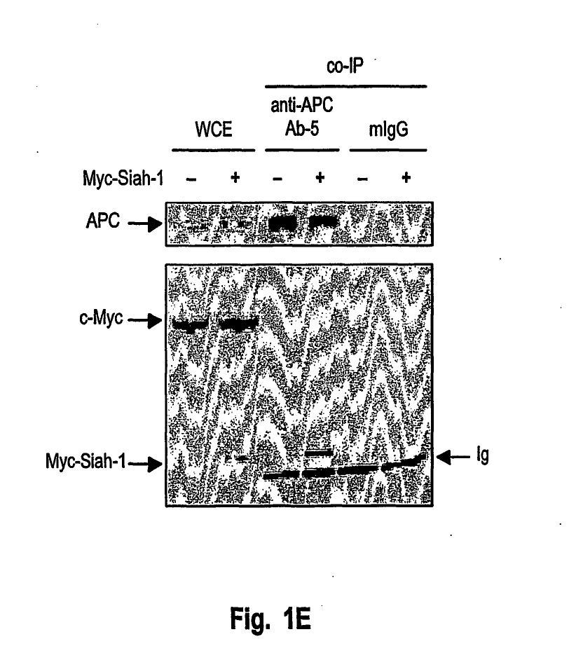

Yeast two-hybrid cDNA library screening identified an interaction between Siah-1 and the carboxy-terminal 155 amino acids of pAPC. To confirm the results of the yeast two-hybrid assay, several Siah-1 and pAPC constructs were tested for in vitro interaction by a GST pull-down assay. In vitro translated .sup.35S-labeled Siah-1 (full-length, a.a. 1-298), .DELTA.N-Siah-1 (the amino-terminal deletion, leaving a peptide with a.a. 97-298) and .DELTA.C-Siah-1 (the carboxy-terminal deletion, which leaves a peptide carrying a.a. 1-193) were tested for binding to various GST-pAPC carboxy-terminal fusion proteins. The results of this test are shown in FIGS. 1A, 1B, and 1C. As in the yeast two-hybrid system, Siah-1 and .DELTA.N-Siah-1, but not .DELTA.C-Siah-1, associated with GST-pAPC carboxy-terminal fusion proteins, indicating that the carboxy-terminal sequences of Siah-1 are needed for binding to pAPC as shown in FIG. 1D. No interaction was observed with GST alone.

To examine whether Siah-1 interacts with pAPC in cells, a plasmid expressing Myc epitope-tagged Siah-1 was transfected into 293T cells and an extract was immunoprecipitated with a mouse monoclonal antibody specific for the amino-terminus of pAPC (Ab-5) or a control mouse IgG. Associated Myc-Siah-1 protein was detected by immunoblotting with an anti-Myc monoclonal antibody. As shown in FIG. 1E, Myc-Siah-1 co-immunoprecipitated with endogenous pAPC.

### Example 2

Siah-1 Down-Regulates Tcf/Lef Reporter Activity

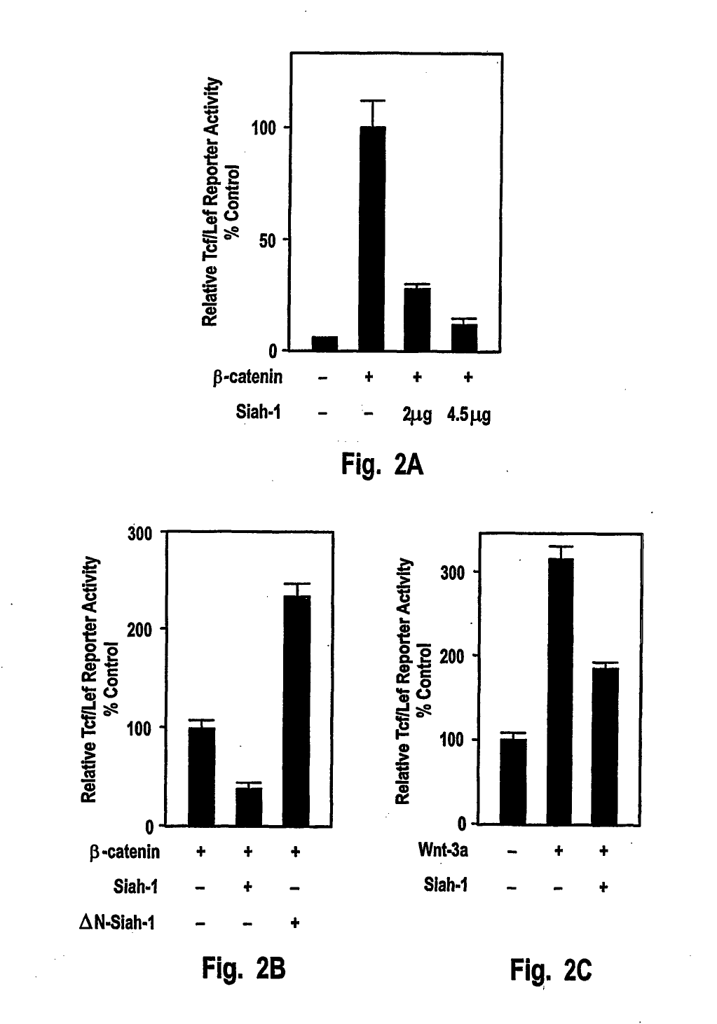

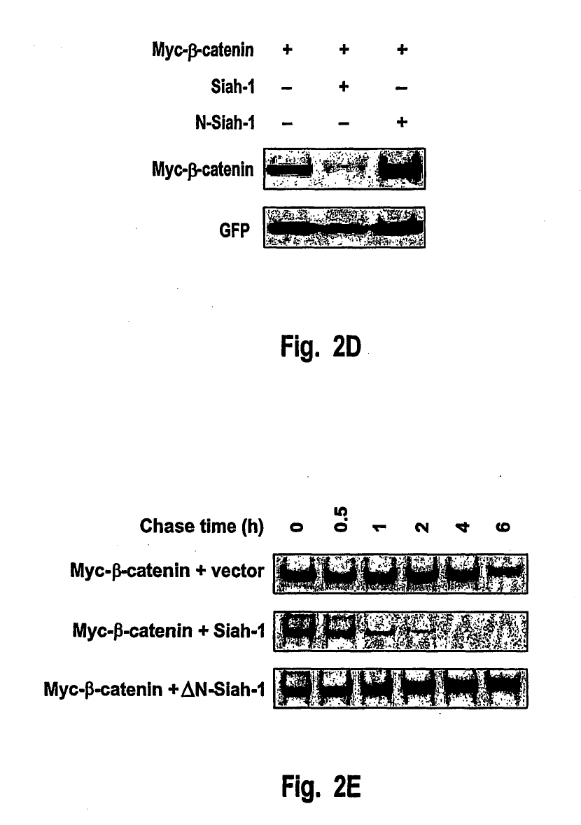

Both Sina and Siah proteins have been implicated in the proteasomal degradation of proteins with which they interact. Li, S., et al. Cell 90: 469478 (1997); Tang, A. H., et al. Cell 90: 459-467 (1997); Hu, G., et al. Genomics 46: 103-111 (1997) Zhang, J., et al. Genes Dev. 12: 1775-1780 (1998). However, initial experiments showed that expression of Siah-1 had no apparent effect on the abundance of pAPC (not shown). Since one function of pAPC is to act as a cytoplasmic scaffold for the assembly of molecules that down-regulate .beta.-catenin, the interaction of Siah-1 with pAPC suggested that Siah-1 might instead regulate .beta.-catenin activity. To test this hypothesis, whether increased expression of Siah-1 would have any effect on Tcf/Lef reporter activity in 293T cells was assessed. Korinek, V., et al. Science 275: 1784-1787 (1997). As shown in FIG. 2A, expression of Siah-1 dramatically decreased the .beta.-catenin-induced Tcf/Lef reporter activity in a dose dependent manner. On the other hand, expression of the amino-terminal deletion mutant, .DELTA.N-Siah-1, increased Tcf/Lef reporter activity as shown in FIG. 2B. The .DELTA.N-Siah-1 protein is known to function as a trans-dominant inhibitor of endogenous Siah-1. Hu, G. & Fearon, E. R. Mol. Cell. Biol. 19: 724-732 (1999). Expression of Siah-1 also decreased Tcf/Lef reporter activity induced by soluble Wnt-3a conditioned medium as shown in FIG. 2C. Shibamoto, S., et al. Genes Cells 10: 659-670 (1998).

### Example 3

Siah-1 Down-Regulates .beta.-catenin

Since the RING domain at the amino-terminus of Siah has been shown to trigger protein degradation, Siah-1 might promote degradation of .beta.-catenin. Hu, G. & Fearon, E. R. Mol. Cell. Biol. 19: 724-732 (1999); Roperch, J. P., et al. Proc. Natl Acad. Sci. USA 96: 8070-8073 (1999). To address this question, Myc-tagged .beta.-catenin was co-expressed in 293T cells with either Siah-1 or .DELTA.N-Siah-1. As shown in FIG. 2D, expression of Siah-1 led to a decrease in the amount of Myc-.beta.-catenin. On the other hand, .DELTA.N-Siah-1 increased the amount of Myc-.beta.-catenin. During these experiments, the levels of c-Myc and I.kappa.B remained unchanged (not shown).

The effect of Siah-1 on .beta.-catenin protein stability was further examined in pulse-chase experiments. Referring to FIG. 2E, Myc-tagged .beta.-catenin was co-expressed with Siah-1 or .DELTA.N-Siah-1 in 293T cells and the rate of Myc-.beta.-catenin turnover was examined. A half-life of several hours was seen when Myc-.beta.-catenin was co-expressed with vector control, consistent with the reported half-life of .beta.-catenin measured in the same cell line. Kitagawa, M., et al. EMBO J. 18: 2401-2410 (1999). Co-expression with Siah-1 reduced the half-life of Myc-.beta.-catenin to approximately 40 minutes. Furthermore, the half-life of Myc-.beta.-catenin was increased when co-expressed with .DELTA.N-Siah-1. These results demonstrate that Siah-1 promotes down-regulation of .beta.-catenin in a post-translational manner and that .DELTA.N-Siah-1 stabilizes .beta.-catenin.

### Example 4

Siah-1 Down-Regulates Mutant .beta.-catenin

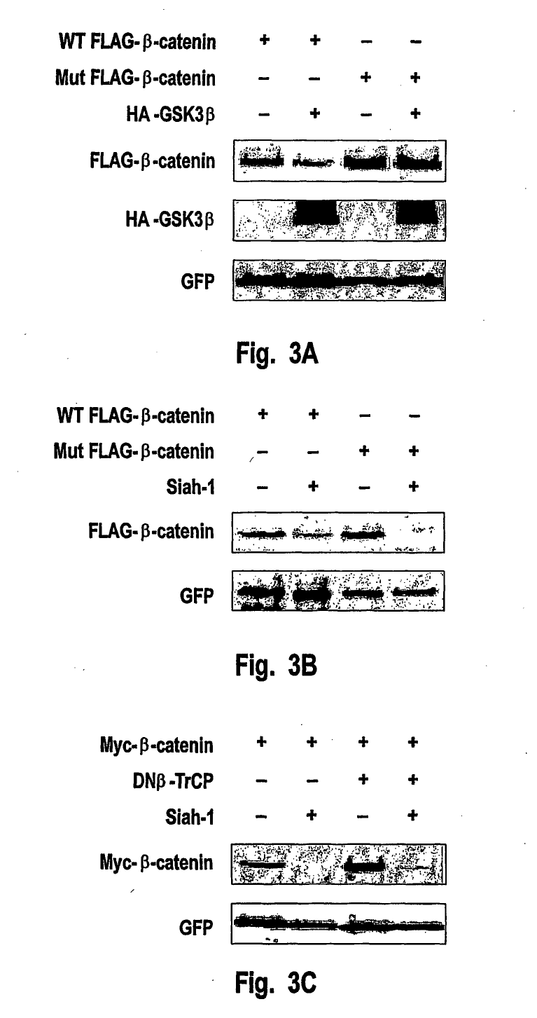

To determine whether Siah-1 regulates the abundance of .beta.-catenin through a mechanism requiring GSK3.beta.-mediated phosphorylation, the effect of Siah-1 was examined on FLAG-tagged .beta.-catenin carrying phenylalanine and alanine substitutions of residues in the putative GSK3.beta. phosphorylation sites (serine and threonine amino acid residues between codons 33 and 45). Such substitutions produce .beta.-catenin protein resistant to phosphorylation by GSK3.beta. and subsequent targeting for proteasome mediated degradation, resulting in increased .beta.-catenin stability. Bienz, M. & Clevers, H. Cell 103: 311-320 (2000); Peifer, M., & Polakis, P. Science 287: 1606-1609 (2000). Consistent with these observations, over-expression of GSK3.beta. led to a decrease in the amount of wild-type FLAG-.beta.-catenin, but had no effect on the amount of mutant FLAG-.beta.-catenin as shown in FIG. 3A. Next Siah-1 was co-expressed with either wild-type or mutant FLAG-.beta.-catenin. As shown in FIG. 3B, expression of Siah-1 led to a decrease in the amount of mutant FLAG-.beta.-catenin as well as wild-type FLAG-.beta.-catenin. This Siah-1-mediated reduction in steady-state levels of wild-type and mutant .beta.-catenin protein was accompanied by an increase in high molecular weight ubiquitin-conjugates of both wild-type and mutant .beta.-catenin in Siah-1-over-expressing cells, as determined by analysis of anti-.beta.-catenin immune-complexes by immunoblotting under denaturing conditions using anti-ubiquitin antibodies (not shown). These results indicate that Siah-1 mediates down-regulation of .beta.-catenin independently of phosphorylation of the amino-terminal serine and threonine residues by GSK3.beta..

### Example 5

Dominant-Negative .beta.-TrCP Does Not Block Siah-1-Mediated Down-Regulation of .beta.-catenin

Since these results indicate that Siah-1 promotes degradation of .beta.-catenin through a mechanism independent of phosphorylation by GSK3.beta., degradation of .beta.-catenin mediated by Siah-1 might occur by a mechanism distinct from the .beta.-TrCP pathway. To test this hypothesis, the .beta.-TrCP pathway was blocked by co-transfecting 293T cells with a plasmid expressing the dominant-negative form of .beta.-TrCP (F-box deletion mutant) together with Myc-tagged wild-type .beta.-catenin. Dominant-negative .beta.-TrCP has been shown to bind phosphorylated .beta.-catenin, but is unable to form an SCF.sup..beta.-TrCP ubiquitin ligase complex, resulting in accumulation of cytoplasmic .beta.-catenin. Hart, M., et al. Curr. Biol. 9: 207-210 (1999). Consistent with these observations, expression of dominant-negative .beta.-TrCP increased the amount of Myc-.beta.-catenin as shown in FIG. 3C, lanes 1 and 3. Co-expression of Siah-1 with dominant-negative .beta.-TrCP, however, dramatically decreased the amount of Myc-.beta.-catenin as shown in FIG. 3C, lanes 3 and 4. Taken together, these results indicate that Siah-1 promotes degradation of .beta.-catenin through a mechanism independent of GSK3.beta.-mediated phosphorylation and the .beta.-TrcP-mediated proteasome pathway.

### Example 6

Siah-1-Mediated Down-Regulation of .beta.-catenin is Dependent on pAPC

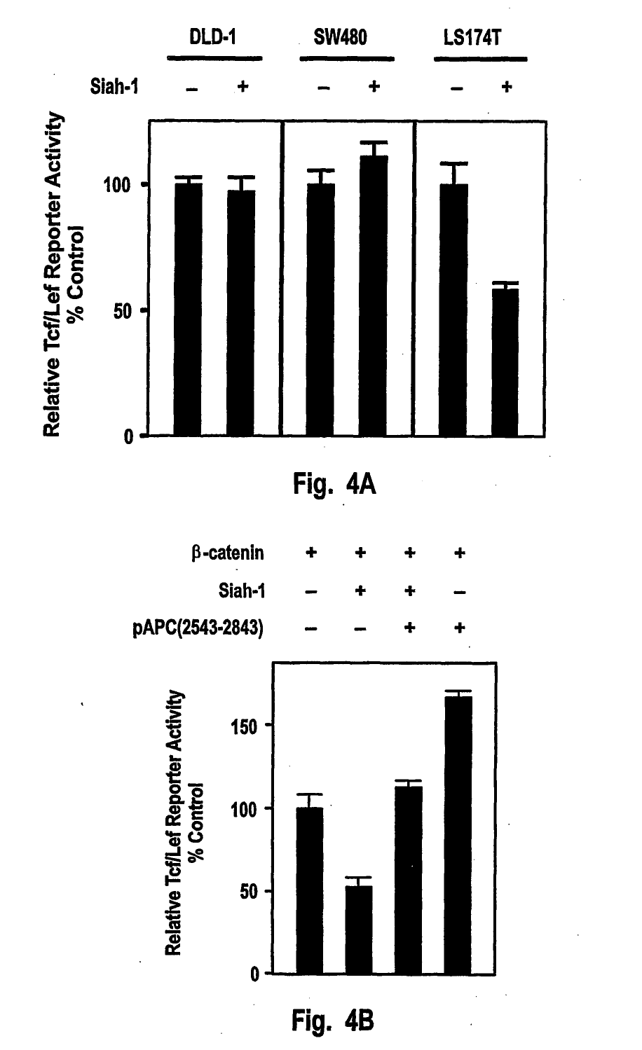

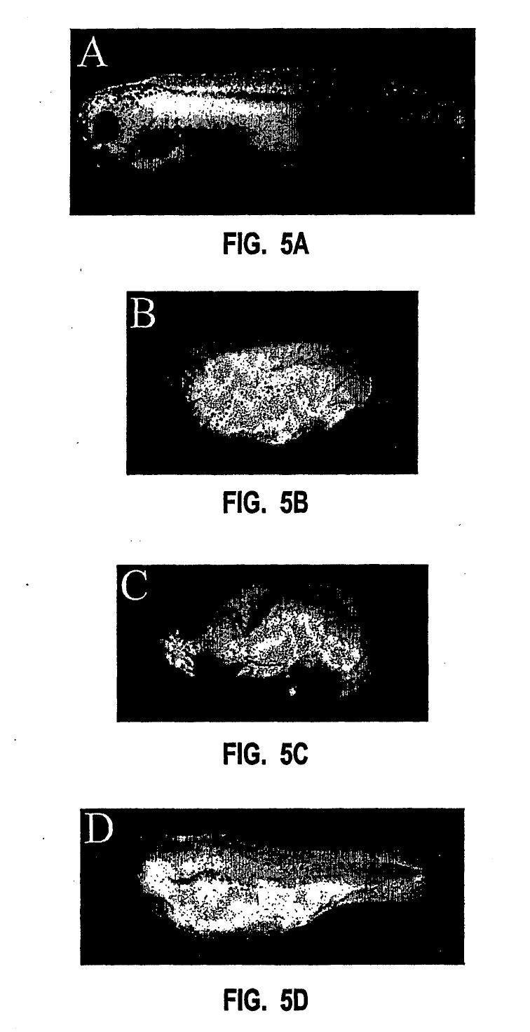

Although Siah-1 bound the carboxy-terminus of pAPC, it was still unclear whether the presence of pAPC was required for Siah-1 to down-regulate .beta.-catenin. To answer this question, the effect of Siah-1 expression on Tcf/Lef reporter activity was tested in the colon cancer cell lines that express truncated and/or wild-type pAPC. As shown in FIG. 4A, expression of Siah-1 failed to decrease Tcf/Lef reporter activity in the colon cancer cell lines which express only truncated pAPC (SW480, DLD1), but Tcf/Lef reporter activity was down-regulated in the colon cancer cell line which expresses a wild-type pAPC (LS 174T). Smith, K. J., et al. Proc. Natl Acad. Sci. USA 90: 2846-2850 (1993). In addition, since LS174T cells express only mutant .beta.-catenin which cannot be phosphorylated by GSK3.beta. (S45F), this observation provides further support for the finding that Siah-1 down-regulates .beta.-catenin independently of GSK3.beta.-mediated phosphorylation. Recently, it has been shown that expression of wild-type pAPC in SW480 cells down-regulates Tcf/Lef reporter activity induced by wild-type .beta.-catenin but not by the non-phosphorylatable mutant .beta.-catenin. Easwaran, V., et al J. Biol. Chem. 274: 16641-16645 (1999). This is due to a low expression level of endogenous Siah-1 transcripts in SW480 cells, with the result that only wild-type .beta.-catenin is down-regulated through the GSK3 .beta. pathway.

Next, it was determined whether over-expression of the carboxy-terminal portion of pAPC can competitively inhibit Siah-l-mediated down-regulation of Tcf/Lef reporter activity in 293T cells. As shown in FIG. 4B, expression of the carboxy-terminal pAPC peptide alone increased Tcf/Lef reporter activity (lanes 1 and 4), a similar effect seen with .DELTA.N Siah-1. Co-expression of the carboxy-terminal pAPC peptide with Siah-1 blocked the down-regulation of Tcf/Lef reporter activity induced by Siah-1 (lanes 2 and 3). These observations indicate that down-regulation of .beta.-catenin by Siah-1 is dependent on pAPC.

### Example 7

Siah-1 Induces Reduced Dorsoanterior Development in Xenopus Embryos

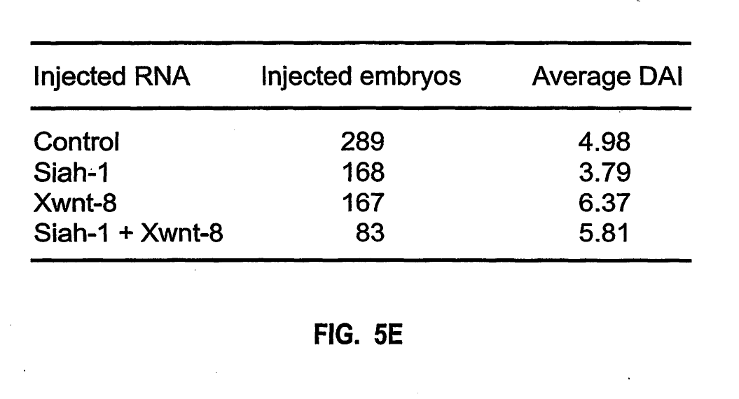

In Xenopus laevis, transcriptional targets of .beta.-catenin include genes that specify the dorsal-ventral axis. To determine whether Siah-1 can regulate transcription of .beta.-catenin target genes in this heterologous system, the effect of Siah-1 expression on dorsoanterior development of Xenopus embryos was examined. As shown in FIG. 5, embryos injected with RNA encoding Siah-1 were ventralized and showed reduced dorsoanterior development, including loss of eyes and reduced head structures. In contrast, embryos injected with RNA encoding Xwnt-8 showed enlarged dorsoanterior structures as reported previously. Smith, W. C. & Harland, R. M. Cell 67: 753-765 (1991). Embryos co-injected with both Siah-1 and Xwnt-8 RNA were fairly normal with eyes and well-developed head structures. These results indicate that Siah-1 can down-regulate .beta.-catenin target genes during the development of Xenopus embryos.

### Example 8

Siah-1 Mediates p53-Induced Degradation of .beta.-catenin

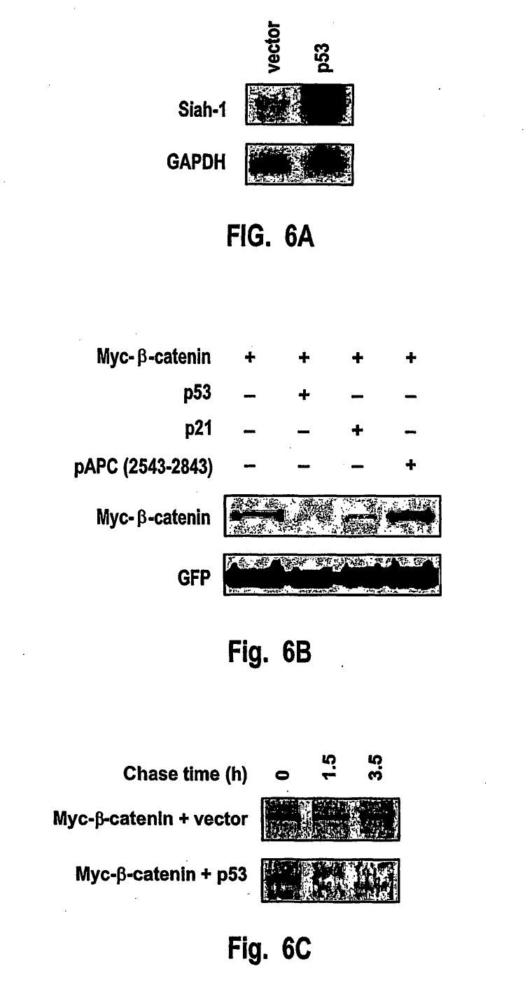

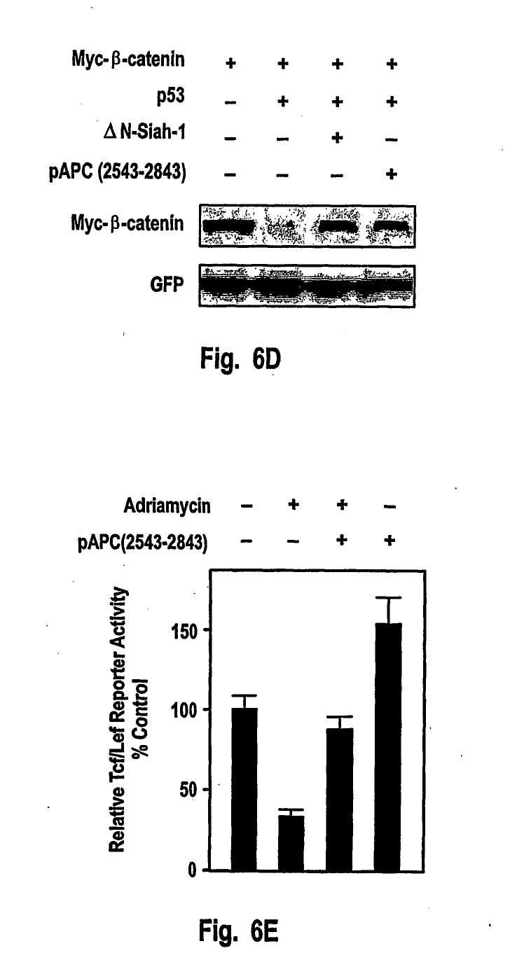

Siah-1 had been reported as a p53-inducible regulator of cell cycle arrest and apoptosis in mammalian cells. Amson, R. B. et al. Proc. Natl Acad. Sci. USA 93: 3953-3957 (1996); Nemani, M. et al. Proc. Natl Acad. Sci. USA 93: 9039-9042 (1996); Hu, G., et al. Genomics 46: 103-111 (1997); Matsuzawa, S., et al. EMBO J. 17: 2736-2747 (1998); Linares-Cruz, G. et al. Proc. Natl Acad. Sci. USA 95: 1131-1135 (1998); Roperch, J. P., et al. Proc. Natl Acad. Sci. USA 96: 8070-8073 (1999). On the other hand, .beta.-catenin promotes the G1 to S phase transition and protects cells from apoptosis. Orford, K., et al. J. Cell Biol. 146: 855-868 (1999). It was determined whether the Siah-1-mediated down-regulation of .beta.-catenin was linked to p53. First, it was confirmed that Siah-1 mRNA is induced by over-expression of p53 in 293T cells by Northern analysis as shown in FIG. 6A. Next, the effect of p53 on .beta.-catenin protein level was examined by transiently over-expressing p53 in 293T cells. The levels of co-transfected Myc-tagged .beta.-catenin was measured by immunoblotting. As shown in FIG. 6B, the level of Myc-.beta.-catenin was dramatically reduced when co-transfected with p53. Similar reduction of Myc-.beta.-catenin levels was seen with the cyclin-dependent kinase inhibitor p21. p21 had previously been shown to induce Siah-1 in mammalian cells. Linares-Cruz, G. et al. Proc. Natl Acad. Sci. USA 95: 1131-1135 (1998); Roperch, J. P., et al. Proc. Natl Acad. Sci. USA 96: 8070-8073 (1999). On the contrary, expression of the carboxy-terminal pAPC peptide stabilized Myc-.beta.-catenin. The level of Tcf/Lef reporter activity corresponded to the amount of Myc-.beta.-catenin measured by immunoblotting (not shown). To further characterize the effect of p53 on reducing .beta.-catenin protein levels, .beta.-catenin protein stability was measured by pulse-chase analysis. Myc-tagged .beta.-catenin was co-expressed with vector control or p53 in 293T cells and the rate of Myc-.beta.-catenin turnover was determined. As shown in FIG. 6C, the half-life of Myc-.beta.-catenin was shortened in the presence of p53, indicating that p53 promotes degradation of .beta.-catenin.

During normal cell cycle, the amount of cytoplasmic .beta.-catenin increases significantly from G1 to S phase. Orford, K., et al. J. Cell Biol. 146: 855-868 (1999). To determine whether .beta.-catenin degradation activated by p53 is secondary to p53-induced cell cycle arrest or directly mediated by p53-induced Siah-1, .DELTA.N-Siah-1 or the carboxy-terminal pAPC peptide was co-expressed with p53 and measured the amount of Myc-.beta.-catenin by immunoblotting. As shown in FIG. 6D, both .DELTA.N-Siah-1 and the carboxy-terminal pAPC peptide attenuated the p53-induced reduction of Myc-.beta.-catenin. This result strongly suggests that .beta.-catenin is a target for protein degradation by p53-induced endogenous Siah-1. During this experiment, the levels of over-expressed p53 protein and induced Siah-1 mRNA were not affected by .DELTA.N-Siah-1 nor the carboxy-terminal pAPC peptide (not shown).

Based on these observations with transient over-expression of p53 and Myc-.beta.-catenin, it was hypothesized that genotoxic injury which activates p53 would down-regulate endogenous .beta.-catenin through induction of endogenous Siah-1. To explore this hypothesis, 293T cells were treated with adriamycin and examined its effect on the amount of endogenous .beta.-catenin by measuring Lef/Tcf reporter activity. Adriamycin has been previously employed to induce p53 protein in 293T cells. Tanikawa, J., et al. J. Biol. Chem. 275: 15578-1558 (2000). As shown in FIG. 6E, adriamycin-treated 293T cells show decreased Tcf/Lef reporter activity. Expression of the carboxy-terminal pAPC peptide blocked the adriamycin-induced decrease of Tcf/Lef reporter activity. Taken together, these observations indicate that APC and Siah-1 mediate a novel .beta.-catenin degradation pathway linking p53 activation to cell cycle control.

Sequence CWU 1

12 1 36 DNA Artificial Sequence Synthetic Oligonucleotide 1 gcgcgcggat ccatggaaaa ggcaaatcca aacatt 36 2 36 DNA Artificial Sequence Synthetic Oligonucleotide 2 ggccggggat ccttaaacag atgtcacaag gtaaga 36 3 36 DNA Artificial Sequence Synthetic Oligonucleotide 3 gcgcgcggat ccatgtcagg aacctggaaa cgtgag 36 4 69 DNA Artificial Sequence Synthetic Oligonucleotide 4 gcgcgcggat ccgccgccac catggactac aaggacgacg atgacaagtc aggaacctgg 60 aaacgtgag 69 5 66 DNA Artificial Sequence Synthetic Oligonucleotide 5 gccagtggta ccgccgccac catggattac aaggatgacg acgataaggc tactcaagct 60 gatttg 66 6 41 DNA Artificial Sequence Synthetic Oligonucleotide 6 acagctatga cctctagatt acaggtcagt atcaaaccag g 41 7 57 DNA Artificial Sequence Synthetic Oligonucleotide 7 aaaaggagct gtggcagtgg caccaaaatg gattccaaag tccaggtaag actgttg 57 8 57 DNA Artificial Sequence Synthetic Oligonucleotide 8 tttggaatcc attttggtgc cactgccaca gctccttttc tgagtggtaa aggcaat 57 9 69 DNA Artificial Sequence Synthetic Oligonucleotide 9 gcgcgcggat ccgccgccac catggactac aaggacgacg atgacaagga cccggccgag 60 gcggtgctg 69 10 36 DNA Artificial Sequence Synthetic Oligonucleotide 10 ggccggtcta gattatctgg agatgtaggt gtatgt 36 11 33 DNA Artificial Sequence Synthetic Oligonucleotide 11 ggccggctcg agagcagtta tgaaatctct ctg 33 12 30 DNA Artificial Sequence Synthetic Oligonucleotide 12 gcgcgcctcg agagaatggt caggacagat 30

1. A method for treating cancer in a human suffering therefrom, comprising administering a compound that mimics Siah-1 activity to a human in need of such treatment.

2. The method of claim 1, wherein the ability of the compound to mimic Siah-l activity is determined by contacting a cell with a test compound, wherein the cell expresses or over-expresses .beta.-catenin and Siah-1 is suppressed in the cell and measuring degradation of .beta.-catenin, wherein a test compound that induces the degradation of .beta.-catenin is a potential cancer chemotherapeutic agent.

3. The method of claim 2, wherein the compound binds to the carboxy-terminus of pAPC.

4. The method of claim 1 wherein the cancer is colorectal cancer.


SEQUENCE LISTING (12 sequences)

SEQ ID NO: 1 - DNA, 36 bp, Artificial Sequence
  Synthetic Oligonucleotide
gcgcgcggatccatggaaaaggcaaatccaaacatt

SEQ ID NO: 2 - DNA, 36 bp, Artificial Sequence
  Synthetic Oligonucleotide
ggccggggatccttaaacagatgtcacaaggtaaga

SEQ ID NO: 3 - DNA, 36 bp, Artificial Sequence
  Synthetic Oligonucleotide
gcgcgcggatccatgtcaggaacctggaaacgtgag

SEQ ID NO: 4 - DNA, 69 bp, Artificial Sequence
  Synthetic Oligonucleotide
gcgcgcggatccgccgccaccatggactacaaggacgacgatgacaagtcaggaacctggaaacgtgag

SEQ ID NO: 5 - DNA, 66 bp, Artificial Sequence
  Synthetic Oligonucleotide
gccagtggtaccgccgccaccatggattacaaggatgacgacgataaggctactcaagctgatttg

SEQ ID NO: 6 - DNA, 41 bp, Artificial Sequence
  Synthetic Oligonucleotide
acagctatgacctctagattacaggtcagtatcaaaccagg

SEQ ID NO: 7 - DNA, 57 bp, Artificial Sequence
  Synthetic Oligonucleotide
aaaaggagctgtggcagtggcaccaaaatggattccaaagtccaggtaagactgttg

SEQ ID NO: 8 - DNA, 57 bp, Artificial Sequence
  Synthetic Oligonucleotide
tttggaatccattttggtgccactgccacagctccttttctgagtggtaaaggcaat

SEQ ID NO: 9 - DNA, 69 bp, Artificial Sequence
  Synthetic Oligonucleotide
gcgcgcggatccgccgccaccatggactacaaggacgacgatgacaaggacccggccgaggcggtgctg

SEQ ID NO: 10 - DNA, 36 bp, Artificial Sequence
  Synthetic Oligonucleotide
ggccggtctagattatctggagatgtaggtgtatgt

SEQ ID NO: 11 - DNA, 33 bp, Artificial Sequence
  Synthetic Oligonucleotide
ggccggctcgagagcagttatgaaatctctctg

SEQ ID NO: 12 - DNA, 30 bp, Artificial Sequence
  Synthetic Oligonucleotide
gcgcgcctcgagagaatggtcaggacagat